(19) United States
(12) Patent Application Publication
(10) Pub. No.: US 2026/0053620 A1
(43) Pub. Date: Feb. 26, 2026

(54) SURGICAL INSTRUMENT

(71) Applicants: KINGLEY RUBBER INDUSTRIAL CO., LTD., New Taipei City (TW); KINGLEY MEDICAL CO., LTD., New Taipei City (TW)

(72) Inventors: Kuan-Cheng SHIH, New Taipei City (TW); Chi-Hsin HUA, New Taipei City (TW); Shih-Chung YANG, New Taipei City (TW)

(21) Appl. No.: 18/810,548

(22) Filed: Aug. 21, 2024

(51) Int. Cl. A61F 2/14; A61F 9/007

(52) U.S. Cl. CPC A61F 2/148; A61F 9/007

(57) ABSTRACT

A surgical instrument including a main body and a push assembly is provided. The main body has an opening. The push assembly includes a pushing component and a driven component. The pushing component is slidably disposed on the main body. The driven component is slidably disposed on the main body and located between the pushing component and the opening, wherein the driven component is adapted to hold an object-to-be-implanted, and the pushing component is adapted to receive a force to slide along the main body and push the driven component, such that the object-to-be-implanted is moved out of the main body through the opening by the driven component.

### BACKGROUND OF THE INVENTION

### (a) Technical Field of the Invention The present invention relates to a surgical instrument, and more particularly to a surgical instrument applicable to an implantation operation.

### (b) Description of the Prior Art

With the development of retinal prosthesis implantation technology, more and more patients with blindness or vision deterioration have improved their vision by implanting prostheses in the retina. For surgical instruments that are commonly used to implant the prosthesis in the retina, it is operated to clamp the prosthesis in order to proceed with implantation of the prosthesis. However, the prosthesis may be broken and damaged due to the clamping force of the surgical instrument. In addition, due to the fact that an eyeball exhibits curvatures, when the surgeon uses surgical instruments to implant the prosthesis into the retina of the eyeball, the surgical instruments may not comply with the curvature of the eyeball and may injure the internal tissues of the eyeball.

### SUMMARY OF THE INVENTION

The present invention provides a surgical instrument, which helps prevent an object-to-be-implanted from getting broken and damaged during an implantation operation and may easily conform to the curvature of the affected part for easy manipulation.

The surgical instrument of the present invention comprises a main body and a push assembly. The main body has an opening. The push assembly comprises a pushing component and a driven component. The pushing component is slidably disposed on the main body. The driven component is slidably disposed on the main body and is located between the pushing component and the open end, wherein the driven component is adapted to hold an object-to-be-implanted, and the pushing component is adapted to receive a force to act thereon so as to slide along the main body and drive the driven component to move to have the driven component move the object-to-be-implanted out of the main body through the opening.

In one embodiment of the present invention, a material of the driven component is different from a material of the pushing component, and the material of the driven component comprises a flexible material.

In one embodiment of the present invention, the main body comprises a first assembly and a second assembly connectable with each other, and the pushing component is slidably on the first assembly and is extendable into the second assembly, and the driven component is slidably disposed in the second assembly, the opening being arranged at an end of the second assembly.

In one embodiment of the present invention, a material of the second assembly is different from a material of the first assembly, and the material of the second assembly comprises a flexible material.

In one embodiment of the present invention, a material of the second assembly comprises rubber or silicone, and a material of the driven component comprises a metal.

In one embodiment of the present invention, the second assembly comprises a coating film provided thereon.

In one embodiment of the present invention, the second assembly comprises a scale arranged thereon.

In one embodiment of the present invention, a surface of the pushing component is formed with a groove, and the groove forms an air flow channel with the main body.

In one embodiment of the present invention, the surgical instrument further comprises a safety latch, wherein the safety latch is insertable into the main body to prevent the pushing component from sliding along the main body.

In one embodiment of the present invention, the surgical instrument further comprises a safety lid, wherein the safety lid is mountable on the main body to cover the opening.

Based on the above, in the surgical instrument according to the present invention, the object-to-be-implanted is held on the driven component of the push assembly and is pushed out of the main body by the push assembly manipulated by an operator to thereby reach a predetermined implantation site of an effected part. In other words, the surgical instrument according to the present invention does not carry out an implantation operation by clamping the object-to-be-implanted and is instead carrying out the implantation operation by pushing forward the object-to-be-implanted, so as to prevent the object-to-be-implanted from being damaged as being broken by a clamping force. Further, the push assembly according to the present invention is not a single one-piece component and is instead divided into the pushing component manipulatable by the operator and the driven component pushable to move by the pushing component.

As such, the driven component can be made of a flexible material that is different from the material of the pushing component, so that during the course of an implantation operation, the driven component can be first bent to a desired shape in order to easily carry out the operation in a condition of conforming to the curvature of the affected part and to reduce the risk of damaging the tissues of the affected part during the operation.

### BRIEF DESCRIPTION OF THE DRAWINGS

FIG. 1 is a perspective view showing a surgical instrument according to an embodiment of the present invention.

FIG. 2 is an exploded view of the surgical instrument shown in FIG. 1.

FIG. 3 is a schematic view illustrating an operation process of the present invention.

FIG. 4 is a schematic view showing the operation process continuing from FIG. 3.

FIG. 5 is a schematic view showing the operation process continuing from FIG. 4.

FIG. 6 is a schematic view showing the operation process continuing from FIG. 5.

FIG. 7 is a schematic view showing the operation process continuing from FIG. 6.

FIG. 8 is a schematic view showing the operation process continuing from FIG. 7.

FIG. 9 is a schematic view showing the operation process continuing from FIG. 8.

FIG. 10 shows an end of a second assembly shown in FIG. 8 located in an affected part.

### DETAILED DESCRIPTION OF THE PREFERRED EMBODIMENTS

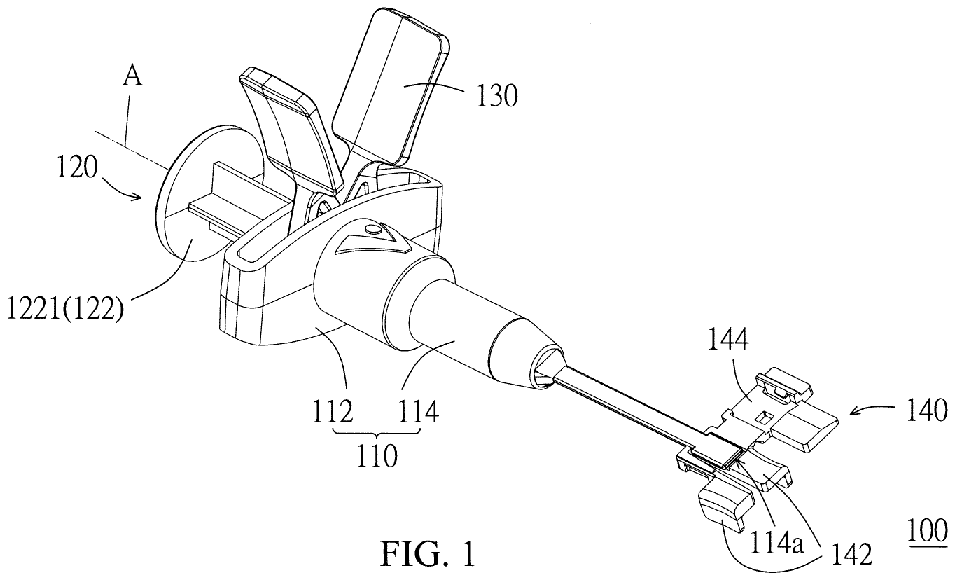

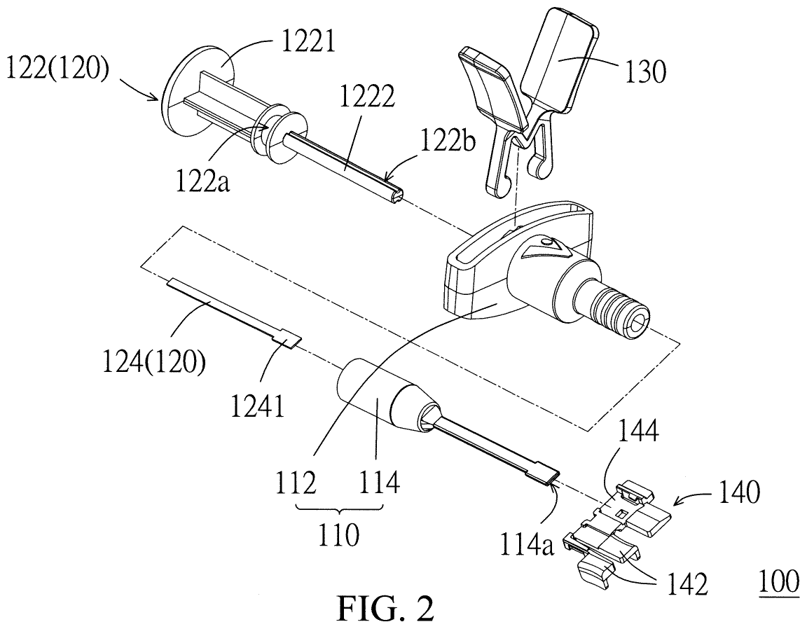

FIG. 1 is a perspective view showing a surgical instrument according to an embodiment of the present invention. FIG. 2 is an exploded view of the surgical instrument shown in FIG. 1. Referring to FIGS. 1 and 2, a surgical instrument 100 according to the instant embodiment comprises a main body 110 and a push assembly 120. The main body 110 has an opening 114a. The push assembly 120 comprises a pushing component 122 and a driven component 124. The pushing component 122 is slidably disposed on the main body 110 in an axial direction A of the main body 110 and comprises a force receiving portion 1221 and a pushing portion 1222. The driven component 124 is also slidably disposed on the main body 110 in the axial direction A of the main body 110, and the driven component 124 is located between the pushing component 122 and the opening 114a. The driven component 124 has an end on which a holding portion 1241 is formed to hold an object-to-be-implanted (such as a retinal prosthesis). The pushing component 122 receives a force acting thereon by means of the force receiving portion 1221 in order to slide along the main body 110 and uses the pushing portion 1222 to push the driven component 124 to move so that the driven component 124 moves the object-to-be-implanted through the opening 114a to get out of the main body 110.

As described above, in the surgical instrument 100 of the instant embodiment, the object-to-be-implanted is held on the driven component 124 of the push assembly 120 and is pushed out of the main body 110 by the push assembly 120 manipulated by an operator to thereby reach a predetermined implantation site (such as the inferior retina of an eyeball) of an affected part (such as the eyeball of a patient). In other words, the surgical instrument 100 according to the instant embodiment does not carry out an implantation operation by clamping the object-to-be-implanted and is instead carrying out the implantation operation by pushing forward the object-to-be-implanted, so as to prevent the object-to-be-implanted C from being damaged as being broken by a clamping force.

Further, in the instant embodiment, the push assembly 120 is not a single one-piece component and is instead split into the pushing component 122 manipulatable by the operator and the driven component 124 pushable to move by the pushing component 122. As such, the driven component 124 can be made of a flexible material that is different from the material of the pushing component 122, so that during the course of an implantation operation, the driven component 124 can be first bent to a desired shape in order to easily carry out the operation in a condition of conforming to the curvature of the affected part and to reduce the risk of damaging the tissues of the affected part during the operation.

In detail, in the instant embodiment, the main body 110 comprises a first assembly 112 and a second assembly 114 that are connectable to each other. The pushing component 122 is slidably disposed on the first assembly 112, while the driven component 124 is slidably disposed in the second assembly 114. The opening 114a is located at an end of the second assembly 114. The pushing component 122 is slidable along the axial direction A of the main body 110 to have the pushing portion 1222 extending into the second assembly 114, in order to push and move the driven component 124 in the way as described above.

Further, in the instant embodiment, in addition to the material of the driven component 124 being selected as a flexible material and different from the material of the pushing component 122, the material of the second assembly 114 is selected as a flexible material and different from the material of the first assembly 112. As such, the second assembly 114, and the driven component 124 disposed therein, can be bent together. For example, the material of the second assembly 114 can be rubber or silicone, and the material of the driven component 124 can be a metal that has excellent ductility and is easy to bend. Further, the materials of the first assembly 112 and the pushing component 122 can be plastics or other suitable materials, and the present invention does not impose limitation thereto.

In the instant embodiment, the surgical instrument 100 further comprises a safety latch 130. The safety latch 130 can be inserted, as shown in FIG. 1, into the first assembly 112 of the main body 110 to engage with and hold a recessed portion 122a (shown in FIG. 2) of the pushing component 122 so as to prevent unexpected sliding of the pushing component 122 along the main body 110. As such, operation safety of the surgical instrument 100 is enhanced. Further, in the instant embodiment, the surgical instrument 100 further comprises a safety lid 140. The safety lid 140 is mounted on the second assembly 114 of the main body 110 to cover the opening 114a, so as to prevent the object-to-be-implanted from unexpectedly dropping out of the main body 110 through the opening 114a. To make the illustration clear, the safety lid 140 is shown in an open condition in FIGS. 1 and 2.

In the following, details of an operation process of the surgical instrument 100 according to the instant embodiment will be provided.

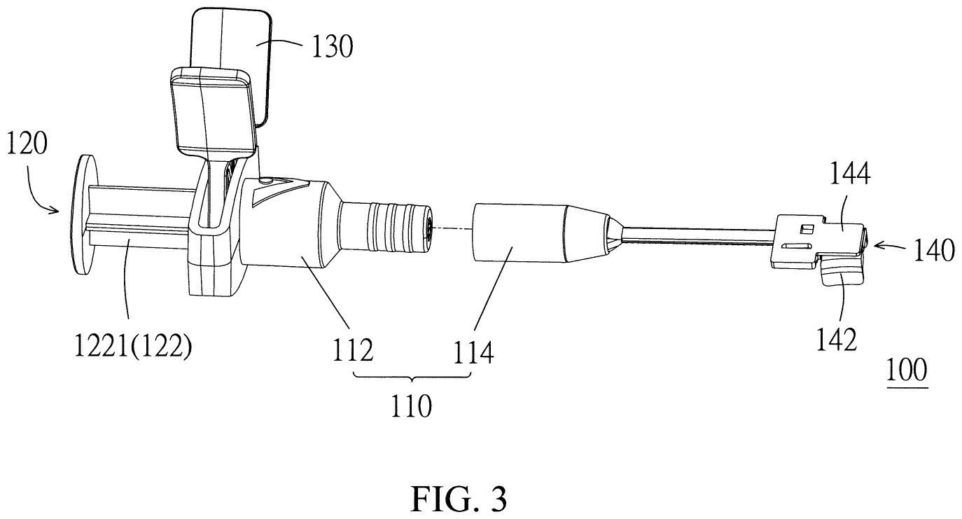

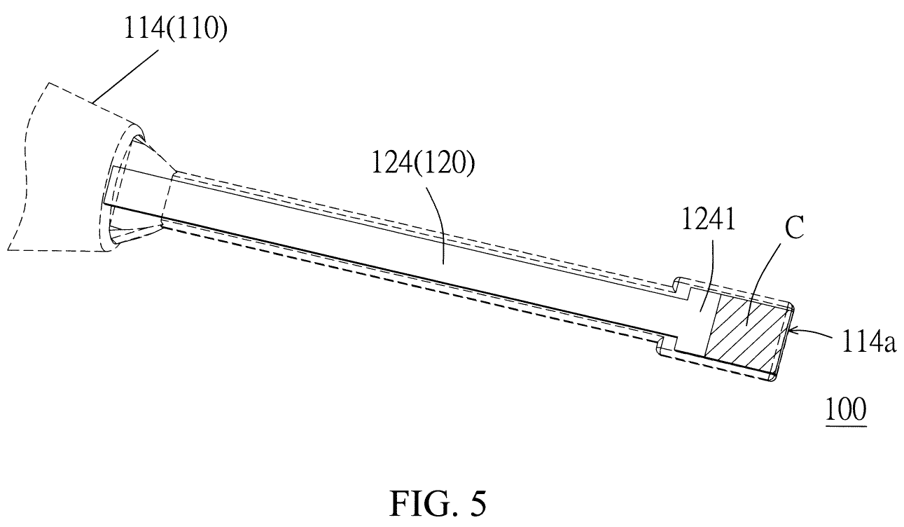

FIGS. 3-9 illustrate a process of operation of the surgical instrument according to an embodiment of the present invention. Firstly, as shown in FIG. 3, a first assembly 112 and a second assembly 114 are provided, wherein the first assembly 112 comprises a pushing component 122 and a safety latch 130, and the second assembly 114 comprises a driven component 124 and a safety lid 140. Then, the first assembly 112 and the second assembly 114 that are shown separated from each other in FIG. 3 are assembled together and an object-to-be-implanted C (shown in FIG. 5) arranged at an end of the second assembly 114 is dipped into water for cleaning. In this step, the second assembly 114 is filled up with water to expel air out to prevent air from entering the affected part in the subsequent operation.

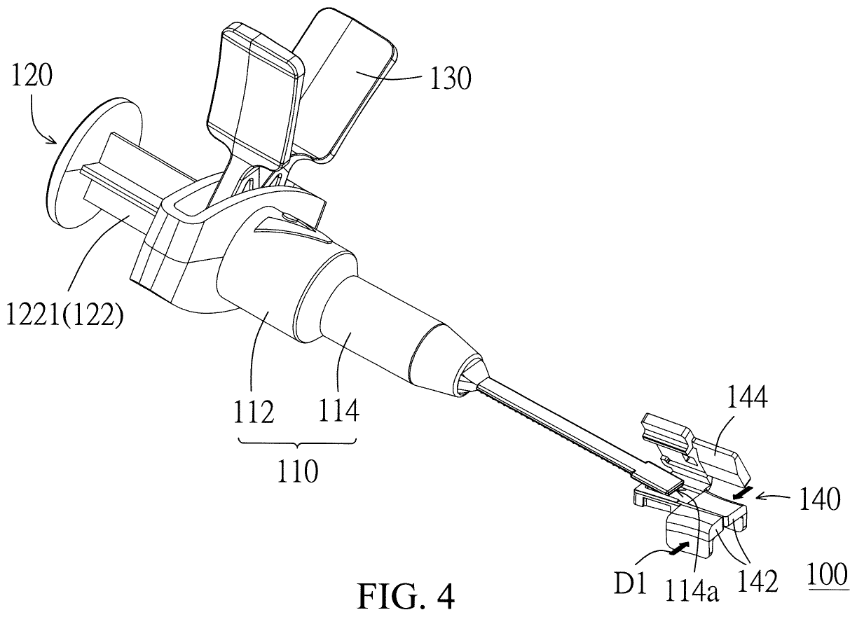

Next, as shown in FIGS. 2, 4, and 5, the safety lid 140 is opened, and a holding portion 1241 at the end of the second assembly 114 is moved away from the safety lid 140. In the instant embodiment, the safety lid 140 can be for example a plastic part that is integrally formed as a one-piece structure, and the user may press a pressing portion 142 in a pressing direction D1 to cause deformation, making a cover portion 144 open as shown in FIG. 4. After the safety lid 140 is opened, confirmation is made as to whether an object-to-be-implanted C is located in the end of the second assembly 114 as shown in FIG. 5, and the second assembly 114 and the driven component 124 disposed therein are bent to conform to the curvature of the affected part to then insert a part of the second assembly 114 into the affected part.

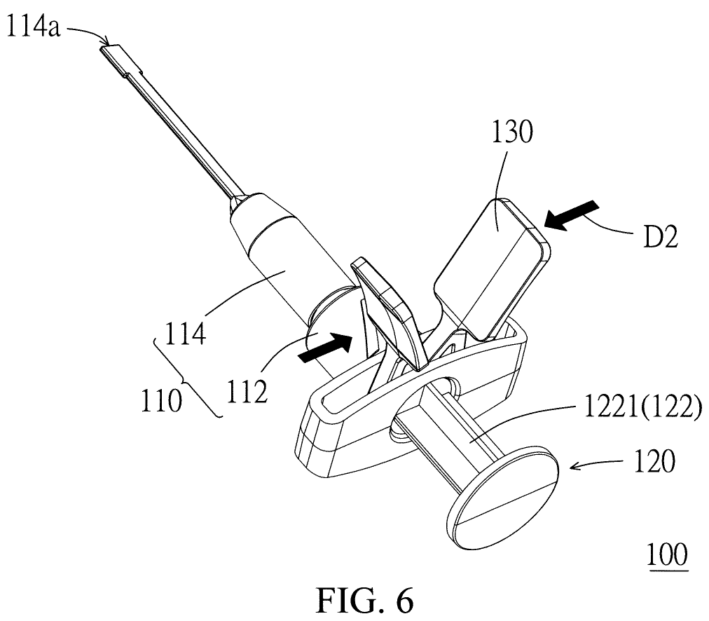

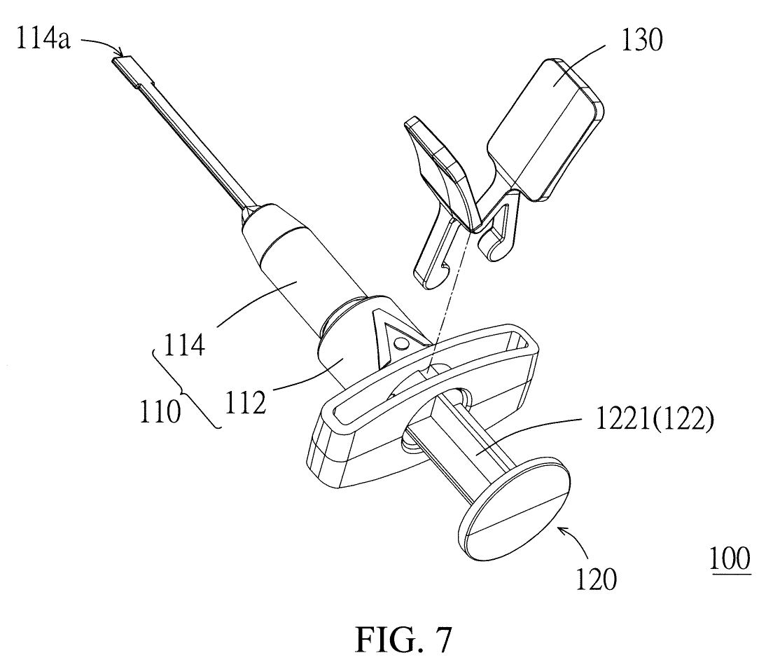

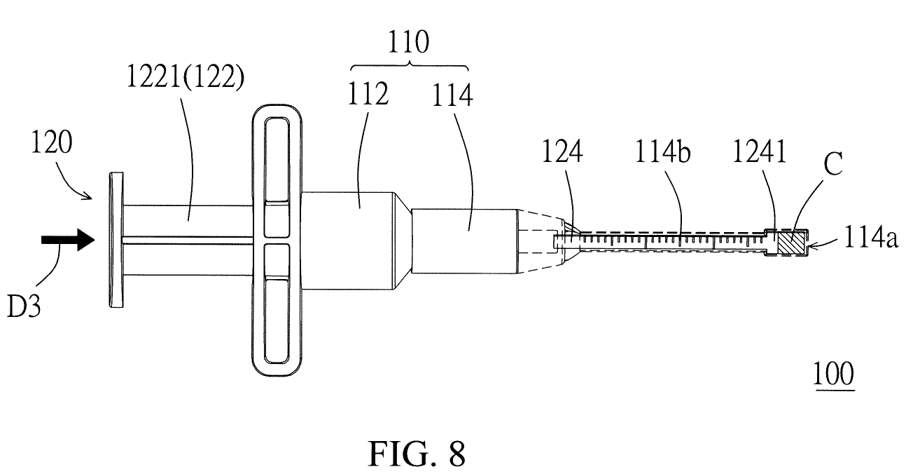

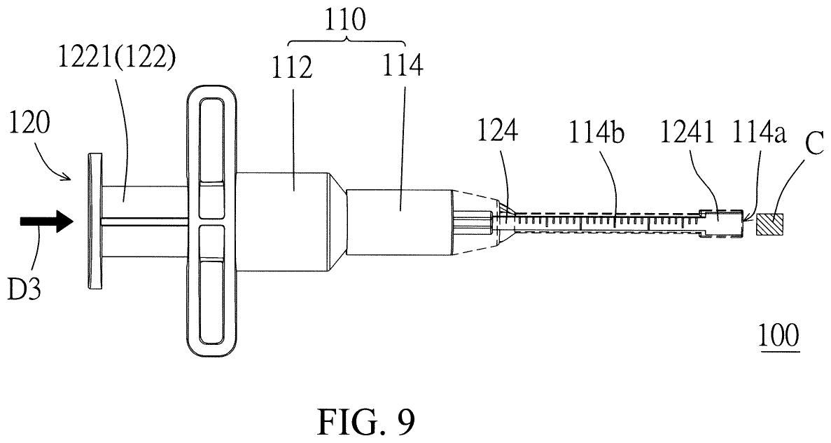

Then, when the end of the second assembly 114 reaches a predetermined implantation site in the affected part, the safety latch 130 is pressed in a pressing direction D2 as shown in FIGS. 6 and 7 to be subsequently moved away from the first assembly 112. Next, as shown in FIGS. 8 and 9, the pushing component 122 is pressed in a pressing direction D3 to allow the pushing component 122 to push and move the driven component 124 to thereby allow the driven component 124 to move the object-to-be-implanted C out of the end of the second assembly 114 to be implanted in the predetermined implantation site in the affected part.

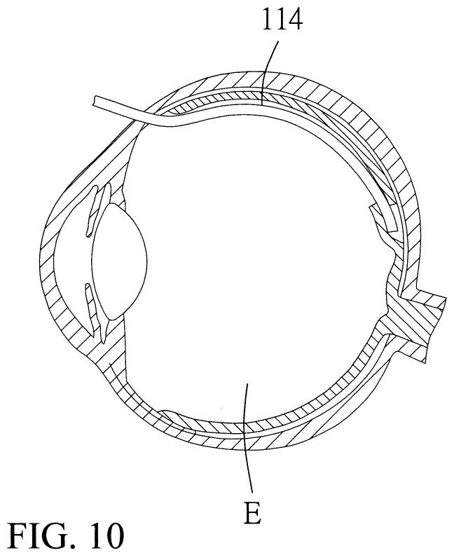

FIG. 10 shows the end of the second assembly shown in FIG. 8 is located in the affected part. Referring to FIG. 10, in detail, when the end of the second assembly 114 gets to the affected part E (the eyeball of the patient), as the second assembly 114 and the driven component 124 has already been bent as described previously, they can extend in a manner of conforming to the curvature of the affected part as shown in FIG. 10. Therefore, the object-to-be-implanted C can be implanted in the predetermined implantation site (inferior retina) in a manner of not causing damage to the affected part E as much as possible.

In the instant embodiment, the second assembly 114 may be provided with a coating film thereon in order to reduce the frictional force between the second assembly 114 and the driven component 124 and the frictional force between the second assembly 114 and the object-to-be-implanted C during the course of the operation described above. Further, the second assembly 114 is provided with a scale 114b as shown in FIGS. 8 and 9, which allows the user to identify the length of the part of the second assembly 114 that extends into the affected part.

Further, in addition to the way of filling water into the second assembly 114 to expel out air therefrom in order to prevent air from entering the affected part during the operation, in the instant embodiment, a groove 122b (shown in FIG. 2) is further formed in a surface of the pushing portion 1222 of the pushing component 122. The groove 122b forms an air flow channel with the first assembly 112 of the main body 110, so that during the process of operation, air may flow through the air flow channel to prevent air from entering the affected part.

In summary, in the surgical instrument of the present invention, the object-to-be-implanted is held on the driven component of the push assembly and is pushed out of the main body by the push assembly manipulated by an operator to thereby reach a predetermined implantation site of an effected part. In other words, the surgical instrument according to the present invention does not carry out an implantation operation by clamping the object-to-be-implanted and is instead carrying out the implantation operation by pushing forward the object-to-be-implanted, so as to prevent the object-to-be-implanted from being damaged as being broken by a clamping force. Further, the push assembly according to the present invention is not a single one-piece component and is instead divided into the pushing component manipulatable by the operator and the driven component pushable to move by the pushing component. As such, the driven component can be made of a flexible material that is different from the material of the pushing component, so that during the course of an implantation operation, the driven component can be first bent to a desired shape in order to easily carry out the operation in a condition of conforming to the curvature of the affected part and to reduce the risk of damaging the tissues of the affected part during the operation.

I claim:

1. A surgical instrument, comprising:
a main body, which has an opening, the main body comprising a first assembly and a second assembly connectable with each other; and
a push assembly, which comprises a pushing component and a driven component, the pushing component being slidably disposed on the main body, the driven component being slidably disposed on the main body and located between the pushing component and the opening, the pushing component being slidably disposed on the first assembly and extendable into the second assembly, the driven component being slidably disposed in the second assembly, the opening being arranged at an end of the second assembly, the pushing component having a surface that is formed with a groove, the groove forming an air flow channel with the main body, the driven component being adapted to hold an object-to-be-implanted, wherein the second assembly and the driven component disposed therein are bendable to conform to curvature of an eye of a patient, and a part of the second assembly is extendable into the eyeball of the patient, such that when the end of the second assembly is moved to reach a predetermined implantation site in the eyeball of the patient, the pushing component is adapted to receive a force to act thereon so as to slide along the main body and drive the driven component to have the driven component moving the object-to-be-implanted outside of the main body through the opening, making the object-to-be-implanted located at the predetermined implantation site in the eyeball of the patient.

2. The surgical instrument according to claim 1, wherein a material of the driven component is different from a material of the pushing component, and the material of the driven component comprises a flexible material.

3. The surgical instrument according to claim 1, wherein a material of the second assembly is different from a material of the first assembly, and the material of the second assembly comprises a flexible material.

4. The surgical instrument according to claim 1, wherein a material of the second assembly comprises rubber or silicone, and a material of the driven component comprises a metal.

5. The surgical instrument according to claim 1, wherein the second assembly comprises a coating film provided thereon.

6. The surgical instrument according to claim 1, wherein the second assembly comprises a scale arranged thereon.

7. The surgical instrument according to claim 1, further comprising a safety latch, wherein the safety latch is insertable into the main body to prevent the pushing component from sliding along the main body.

8. The surgical instrument according to claim 1, further comprising a safety lid, wherein the safety lid is mountable on the main body to cover the opening.
